(19) United States
(12) Patent Application Publication
(10) Pub. No.: US 2026/0076618 A1
(43) Pub. Date: Mar. 19, 2026

(54) System and Methods for Monitoring Caloric Intake and Weight Loss

(71) Applicant: Diamond Distributors, Inc., New York, NY (US)

(72) Inventor: Eli Nhaissi, Westbury, NY (US)

(73) Assignee: Diamond Distributors, Inc., New York, NY (US)

(21) Appl. No.: 18/886,116

(22) Filed: Sep. 16, 2024

(51) Int. Cl. A61B 5/00; A61B 5/02; A61B 5/021

(52) U.S. Cl. CPC A61B 5/4833; A61B 5/02028; A61B 5/021; A61B 5/681; A61B 5/7225; A61B 5/7264; A61B 5/7271; A61B 5/7405; A61B 5/742; A61B 5/746; A61B 2560/045; A61B 2562/0252

(57) ABSTRACT

A wireless network includes: a mobile device that includes a first central processing unit (CPU), a memory, and a first wireless communication module; a first instrument that includes a second CPU, a physiological sensor, and a second wireless communication module paired with the first wireless communication module; and a second instrument that includes a third CPU, a load sensor, and a third wireless communication module paired with the first wireless communication module. The second CPU is programmed to cause digital physiological variable data derived from analog data output by the physiological sensor to be broadcast by the second wireless communication module. The third central processing unit is programmed to cause digital body weight data derived from analog data output by the load sensor to be broadcast by the third wireless communication module. The first CPU is programmed to convert the digital physiological variable data into digital caloric intake data in accordance with an empirical relationship of a physiological variable to caloric intake stored in the memory and store the digital body weight data in the memory.

### BACKGROUND

This invention relates to a system and a method for managing a dietary program. In particular, it relates to a system, methods, and instruments for monitoring caloric intake and weight loss of a dieting individual. As used herein, the noun “diet” means a regimen of eating and drinking sparingly so as to reduce one's weight; the verb “to diet” means to eat and drink sparingly according to prescribed rules; and “sparingly” means marked by or practicing careful restraint (as in the use of resources).

Hundreds of dieting techniques have been proposed based on different and varied principles, some sound and others not so sound and even damaging. The fundamental principle on which every sound method of dieting is based is that, in order to lose weight, the caloric intake must be less than the energy output of the individual.

However, this relationship between the expenditure and the intake of energy is asymmetric and thus, for example, in order to expend the caloric intake added to the body consequent to eating a single slice of bread, it would be necessary for the individual to walk approximately one kilometer. The conclusion, therefore, is that increasing the energy expenditure, by means of exercise, is not itself sufficient to lose weight and that limiting the caloric intake by eating less is essential. However, such a formula is problematic because the amount of food which is consumed is a function of appetite and even hunger of the individual.

One method of controlling the eating behavior of a dieter was disclosed in U.S. Pat. No. 5,398,688. The disclosed method was based on the various physiological changes (such as cardiovascular changes) and processes which occur in the human body during eating. For example, along with the well-known increases in the secretion of salivary juices and of various enzymes, major alterations also take place in the cardiovascular system comprising the individual's heart and blood vessels. The most noticeable changes in the cardiovascular system during the process of eating are a rise in the heart rate and cardiac output and a lowering of the resistance of the peripheral blood vessels. As a result of experimentation, it was found that the rise in the cardiac output is not dependent only on the rise in pulse rate, but also results from an increase in the heart stroke volume. (Stroke volume is the amount of blood ejected from the ventricle with each cardiac cycle. Multiplying the stroke volume by the heart rate yields the cardiac output, typically reported in liters per minute.)

The changes in the cardiovascular system are a result of the processes which occur in the digestive, circulatory, and nervous systems during eating. The specific magnitude of the response and its nature depend on the caloric value of the meal and its constituents (i.e., food categories, proteins, fats and carbohydrates) and the speed of eating. The sensitivity of the reaction of the cardiovascular system is individual and varies greatly from one person to the next.

### SUMMARY

The subject matter disclosed in some detail below is directed to a system, methods, and instruments for monitoring caloric intake and weight loss, during management of a dietary program designed to produce weight loss. The instruments utilized include a wristwatch having a physiological sensor connected to a first central processing unit (hereinafter “CPU”), a body weight scale having a load sensor connected to a second CPU, and a mobile device (e.g., a mobile telephone or a personal digital assistant) having a third CPU configured to execute a diet management application. As used herein, the term “application” means a computer program that is designed to carry out a specific task other than one relating to the operation of the computer itself, typically to be used by end-users. As used herein, the verb “connected” means communicatively coupled directly without an intervening component or indirectly by way of an intervening component. For example, a sensor may be “connected” to a CPU by way of an intervening analog-to-digital converter (hereinafter “ADC”); a memory may be “connected” to a CPU by way of an intervening memory management unit (hereinafter “MMU”); and an antenna may be connected to a broadband mobile communications chip by way of an intervening power amplifier.

The diet management application includes a user-selectable caloric intake monitoring routine that is capable of monitoring physiological variable data output by the wristwatch during an eating occurrence and a user-selectable analysis routine that is capable of computing the amount of weight loss based on body weight data periodically output by the body weight scale.

The proposed method for monitoring caloric intake during management of a dietary program employs the basic process of: selecting a calorie target for each type of meal (e.g., breakfast, lunch, dinner, and snack) based on a weight loss target; computing the caloric intake during each meal of a particular type based on an empirical relationship (predetermined for the particular dieting individual during pre-diet monitoring) between a change in a physiological variable (e.g., a cardiovascular variable) and a rate and/or amount of caloric intake; and then generating an indication that the dieting individual should stop eating when the amount of caloric intake during that meal is equal to or greater than the preset calorie target for that particular type of meal. The empirical relationship may be the same for different types of meals or respective empirical relationships may be determined for the different types of meal.

In accordance with some embodiments, a physiological variable (e.g., a cardiovascular variable) of the dieting individual is monitored during each eating occasion (e.g., breakfast, lunch, dinner, and snacking) using a physiological sensor that may be incorporated in a wristwatch that has a short-range wireless communication capability. A central processing unit (hereinafter “CPU”) in the wristwatch is configured to receive digital data representing the analog physiological variable data output by the physiological sensor and then transmit the physiological variable data in digital format in accordance with the protocol employed by its wireless communication module.

In accordance with a further aspect of the monitoring method proposed herein, the weight of the dieting individual is measured at regular intervals of time (e.g., daily) using a body weight scale having a load sensor and a short-range wireless communication capability. A CPU in the body weight scale is configured to receive digital data representing the analog load sensor data output by the load sensor, convert the load sensor data into body weight data, and then transmit the body weight data in digital format in accordance with the protocol employed by its wireless communication module.

Preferably, the wireless communication modules of the wristwatch and the body weight scale use the same short-range communications protocol. Both the physiological variable data acquired by the wristwatch and the body weight data computed by the body weight scale are time-stamped.

In accordance with a further aspect of the monitoring system proposed herein, the short-range wireless communication modules of both the wristwatch and the body weight scale are paired with the short-range wireless communication module of a mobile device (e.g., a mobile telephone or a personal digital assistant) to enable the transmission of acquired data from the instruments (i.e., wristwatch and scale) to the mobile device and the transmission of control signals or programming from the mobile device to the instruments. (As used herein, “pairing” means a form of information registration for linking devices.) In accordance with one embodiment, a CPU of the mobile device executes a caloric intake monitoring routine that converts the physiological variable data received from the wristwatch into caloric intake data during each eating occasion. The caloric intake data in turn is stored in a non-transitory tangible computer-readable storage medium (hereinafter “memory”) which is incorporated in the mobile device. The memory also stores body weight data received from the body weight scale. In addition, the CPU of the mobile device may be selectively activated to execute an analysis routine that computes the amount of weight loss during a most recent interval of time. In response to a determination that the computed weight loss is less than a preset weight loss target, the CPU 54 is capable of reducing the magnitudes of the calorie targets stored in memory 68 for each type of eating occurrence, which revised calorie targets will be used to monitor succeeding eating occurrences. The mobile device is further equipped with a wireless local area network (WLAN) module to enable the mobile device to communicate with a remote device via the Internet. For example, the mobile device may transmit the caloric intake data and the body weight data via the Internet to a remote device used by a dietician who is responsible for overseeing the individual's dietary program.

Pursuant to implementation of a dietary program, the individual who is planning to diet must download a diet management application to his mobile device from a server in the cloud. Then the individual must pair his sensor-equipped wristwatch with his application-loaded mobile device and also pair the body weight scale which he intends to use with his application-loaded mobile device. For example, each of the wristwatch, body weight scale, and mobile device can incorporate a chip that provides Bluetooth capability. Bluetooth is a short-range wireless technology standard that is used for exchanging data between fixed and mobile devices over short distances and building personal area networks.

Each time that the dieting individual uses the body weight scale, he may input a command that causes the body weight scale to transmit the body weight data to the mobile device. Alternatively, the body weight scale may be programmed to automatically transmit the body weight data upon the conclusion of each weighing event.

In accordance with another embodiment, before the start of each eating occurrence (e.g., meal or snack), the dieting individual first activates a measurement routine on his wristwatch. Then the dieting individual activates the caloric intake monitoring routine on his mobile device prior to starting to eat. As part of this activation process, the dieting individual inputs into the mobile device data identifying the type of eating occurrence (breakfast, lunch, dinner, or snack) which is about to begin. While the dieting individual is eating, the CPU of the wristwatch executes the measurement routine and then transmits acquired physiological variable data to the mobile device; and the CPU of the mobile device processes the physiological variable data from the wristwatch to determine the rate of caloric intake while also tracking the time elapsed since the start of eating. The CPU of the mobile device then computes a maximum allowable eating time based on the rate of caloric intake and a calorie target retrieved from memory for the particular type of meal being eaten. The calorie targets for different categories of eating occurrence may be selected by a dietician responsible for the design of a dietary program recommended to achieve a target weight loss. When the elapsed time of the eating occurrence becomes equal to or greater than the maximum allowable eating time, the mobile device issues an audio or visual alarm indicating that the dieting individual should stop eating. This monitoring process may be repeated for each eating occurrence until the weight loss target has been achieved.

The diet management application installed in the mobile device further includes an analysis routine which computes the current weight loss of the dieting individual and then compares the current weight loss to a target weight loss. In the absence of any weight loss or insufficient weight loss, the analysis routine automatically reduces the magnitudes of the calorie targets stored in memory, which reduced calorie targets are then used to monitor subsequent eating occurrences. In the event that a determination is made that the weight loss target has been achieved, the mobile device generates an audible or visual indication notifying the dieting individual of such successful outcome.

Although various embodiments of a system and methods for monitoring caloric intake and weight loss will be described in some detail below, one or more of those embodiments may be characterized by one or more of the following aspects.

One aspect of the subject matter disclosed in detail below is a wireless network comprising a first instrument and a mobile device. The mobile device comprises a first central processing unit, a memory connected to the first central processing unit, a first wireless communication module connected to the first central processing unit, and a first antenna connected to the first wireless communication module. The first instrument comprises a second central processing unit, a first analog-to-digital converter connected to the second central processing unit, a physiological sensor connected to the first analog-to-digital converter, a second wireless communication module connected to the second central processing unit and paired with the first wireless communication module, and a second antenna connected to the second wireless communication module. The second central processing unit is programmed to cause digital physiological variable data derived from analog data output by the physiological sensor to be broadcast by the second wireless communication module during an eating occurrence of a dieting individual. The first central processing unit is programmed to convert the digital physiological variable data into digital caloric intake data in accordance with an empirical relationship of a physiological variable to caloric intake stored in the memory.

In accordance with some embodiments, the wireless network described in the immediately preceding paragraph further comprises a second instrument. The second instrument comprises a third central processing unit, a second analog-to-digital converter connected to the third central processing unit, a load sensor connected to the second analog-to-digital converter, a third wireless communication module connected to the third central processing unit and paired with the first wireless communication module, and a third antenna connected to the third wireless communication module. The third central processing unit is programmed to cause digital body weight data derived from analog data output by the load sensor to be broadcast by the third wireless communication module. The first central processing unit is further programmed to: store the digital body weight data in the memory of the mobile device; compute an amount of weight loss during a most recent interval of time; determine that the computed weight loss is less than a preset weight loss target stored in the memory of the mobile device; reduce a magnitude of a calorie target stored in the memory of the mobile device to produce a revised calorie target; and use the revised calorie target to monitor caloric intake during a subsequent eating occurrence.

Another aspect of the subject matter disclosed in detail below is a mobile device having components and features as described in the preceding two paragraphs.

A further aspect of the subject matter disclosed in detail below is a method for monitoring caloric intake during an eating occurrence using a mobile device, the method comprising: (a) storing an empirical relationship of a physiological variable to a caloric intake of a dieting individual in a memory of the mobile device; (b) activating a measurement routine to measure a cardiovascular variable of the dieting individual during eating using a first instrument; (c) activating a caloric intake monitoring routine on the mobile device; (d) establishing a first communication channel between the first instrument and the mobile device; (e) transmitting cardiovascular variable data from the first instrument to the mobile device via the first communication channel while the dieting individual is eating; (f) processing the cardiovascular variable data in the mobile device by integrating the cardiovascular variable with respect to elapsed time during the eating occurrence; (g) converting the cardiovascular variable data into caloric intake data in accordance with the empirical relationship; (h) determining that a caloric intake has exceeded a calorie target; and (i) issuing an audio or visual alarm from the mobile device indicating that the dieting individual should stop eating.

In accordance with some embodiments, the method described in the immediately preceding paragraph further comprises: (j) activating a measurement routine to measure a body weight of the dieting individual using a second instrument; (k) establishing a second communication channel between the second instrument and the mobile device; (l) transmitting body weight data from the second instrument to the mobile device via the second communication channel; (m) storing the digital body weight data in a memory of the mobile device; (n) computing an amount of weight loss during a most recent interval of time; (o) determining that the computed weight loss is less than a preset weight loss target; (p) reducing a magnitude of the calorie target to produce a revised calorie target; and (q) using the revised calorie target to monitor caloric intake during a subsequent eating occurrence.

Other aspects of a system and methods for monitoring caloric intake and weight loss are disclosed below.

### BRIEF DESCRIPTION OF THE DRAWINGS

The features, functions and advantages discussed in the preceding section can be achieved independently in various embodiments or may be combined in yet other embodiments. Various embodiments will be hereinafter described with reference to drawings for the purpose of illustrating the above-described and other aspects. None of the diagrams briefly described in this section are drawn to scale.

FIG. 1 is a block diagram identifying some components of a system for monitoring caloric intake and weight loss at a remote location in accordance with one embodiment.

FIG. 2 is a flowchart showing the principal steps of a method for monitoring caloric intake.

FIG. 3 is a graphical representation showing the effects of eating on heart rate.

FIG. 4 is a graphical representation showing the effects of eating on stroke volume.

FIG. 5 is a flowchart showing the principal steps associated with a monitoring phase.

FIG. 6 is a flowchart showing the principal steps associated with a process that monitors the cumulative caloric intake of a dieting individual based on measured changes in a cardiovascular variable in accordance with one embodiment.

FIG. 7 is a flowchart showing the principal steps associated with an analytic process that concludes with an adjustment of the calorie target based on measured changes in body weight in accordance with one embodiment.

FIG. 8 is a flowchart showing the principal steps associated with a monitoring process that monitors the rate of caloric intake of a dieting individual based on measured changes in a cardiovascular variable in accordance with another embodiment.

FIG. 9 is a block diagram identifying some components of a monitoring instrument in accordance with one embodiment for carrying out the caloric intake monitoring methods disclosed herein.

FIG. 10 is a pictorial representation of an integrated monitoring instrument for carrying out the caloric intake monitoring methods disclosed herein.

FIG. 11 is a pictorial representation of a detail of the instrument depicted in FIG. 10.

FIG. 12 is a block diagram identifying some components of a body weight scale in accordance with one embodiment.

FIG. 13 is a block diagram identifying some components of a mobile device in accordance with one embodiment.

Reference will hereinafter be made to the drawings in which similar elements in different drawings bear the same reference numerals.

### DETAILED DESCRIPTION

Illustrative embodiments of systems and methods for monitoring caloric intake and weight loss of a dieting individual are described in some detail below. However, not all features of an actual implementation are described in this specification. A person skilled in the art will appreciate that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system disclosed in detail below provides a framework for dieting which may include an institution or workshop for supervising and appraising the various monitoring activities of an individual who has been enrolled in a dietary program. In accordance with one such dietary program, each prospective dieter is given a non-invasive medical examination in the institution in order to determine his specific cardiovascular reaction throughout three normal or average meals during the course of a day. For example, the food components of each meal may be selected to provide respective target calorie levels for breakfast, lunch, dinner, and snacks consistent with the levels set by the Committee to Review Child and Adult Care Food Program Meal Requirements (Institute of Medicine) in 2011 for the age group to which the particular dieting individual belongs. For example, in the case of a dieting individual in the age group 19-59 years, the target calorie level is 440 kcal for breakfast, 640 kcal for lunch, 640 kcal for dinner, and 140 kcal for snack. The amount of food and its composition are varied during the different meals in order to provide as much information as possible on the various possible reactions of the prospective dieter under examination. This initial pre-diet monitoring process is computerized and continues for a week or more in order to increase the statistical likelihood that the observed reactions are reliable and reproducible. Analysis of the results permits a fairly good prediction of the expected cardiovascular reaction during a meal, with respect to the specific individual under examination and, conversely, determination of the cardiovascular reaction permits assessment of the amount of food eaten (e.g., the caloric intake) during a meal. Furthermore, this is done without the appraiser being present during the eating process. Instead, the appraisal is based only on the analysis of data which has already been recorded and stored in a computer.

In order to derive a reliable correlation of cardiovascular function and caloric intake, each prospective dieter must exercise sufficient self-control and discipline, since different people are able to exhibit recognized variations in their body reactions to an identical meal. The initial monitoring is preferably carried out over a period of approximately one to two weeks in order to ensure that, despite such variations which are influenced by different types of meal, meal times and so on, the average provides for a sufficiently accurate baseline to permit satisfactory control of the prospective dieter's eating habits.

Based on the data acquired during the initial pre-diet monitoring phase, an empirical relationship for the individual between a change in at least one physiological variable (e.g., a cardiovascular variable) and a rate and/or amount of caloric intake is established and stored in computer memory. In accordance with one embodiment, a first empirical relationship may be established for breakfasts, a second empirical relationship may be established for lunches, a third empirical relationship may be established for dinners, and a fourth empirical relationship may be established for snacks in the event that these empirical relationships are significantly different. In accordance with another embodiment, one empirical relationship may be established for all meals if the empirical relationship is relatively constant across the four categories of meals. Prior to the start of the dietary program, digital data representing the empirical relationship(s) is downloaded to the dieting individual's mobile device and stored in memory for use during future activations of the caloric intake monitoring process executed by the mobile device. In the event that first through fourth meal-specific empirical relationship(s) are stored in the memory of the mobile device, the CPU of the mobile device is configured (i.e., programmed) to retrieve and employ the empirical relationship that corresponds to the category of the meal to be eaten by the dieting individual. For example, in response to an input by the dieting individual that the meal to be monitored is a dinner, the CPU of the mobile device will employ the third empirical relationship established for dinners during the caloric intake monitoring process.

In addition, the institution will select appropriate calorie targets for the monitored individual to achieve a target weight loss. Typically, the selected calorie target will be a predetermined percentage less than the aforementioned target calorie levels for breakfast, lunch, dinner, and snacks recommended by the Institute of Medicine. Prior to the start of the dietary program, digital data representing the individual-specific calorie targets is downloaded to the dieting individual's mobile device and stored in memory for use during future activations of the caloric intake monitoring process executed by the mobile device. During each monitored meal of a particular type, the cumulative caloric intake computed by the CPU of the mobile device will be compared to the corresponding calorie target for that particular meal type.

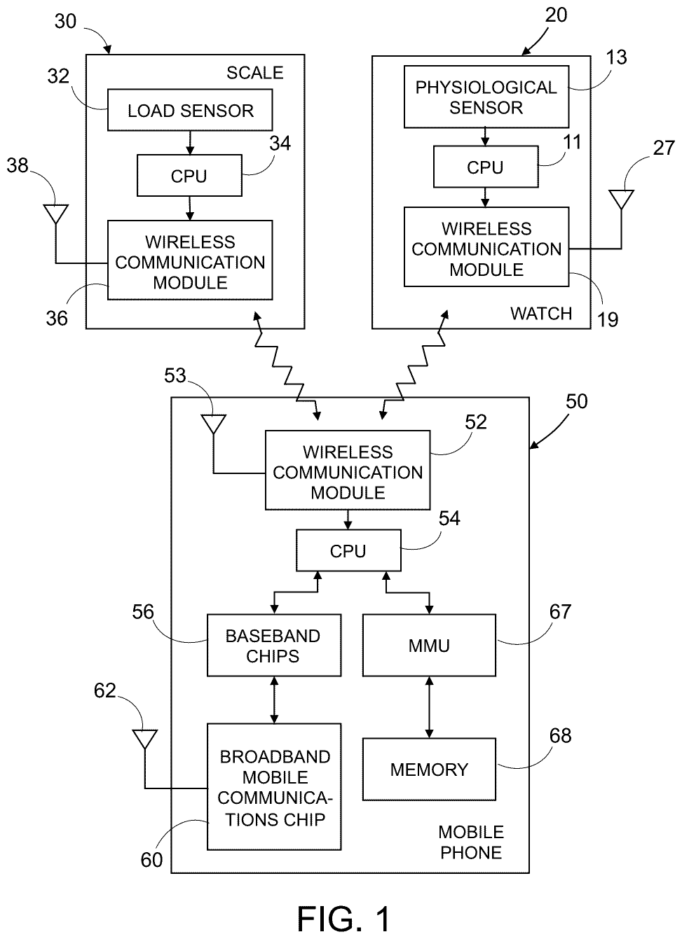

FIG. 1 shows a system comprising a wristwatch 20, a body weight scale 30, and a mobile telephone 50 arranged to form a wireless data communication network. wristwatch 20, body weight scale 30, and mobile telephone 50 each have wireless data communication capability. Wireless communication modules 19, 36, and 52, of wristwatch 20, body weight scale 30, and mobile telephone 50 respectively, may include one or more of Wi-Fi connections, Bluetooth connections, GSM or CDMA cellular communications, or other wireless protocols. The system components depicted in FIG. 1 are programmed to provide a methodology to enable a dieting individual to track the individual's body weight and caloric intake over time. The system may also include a data network (not shown) such as the Internet, which allows the mobile telephone 50 to send and receive social media messages, as well as to store body weight and caloric intake data at a server in the cloud.

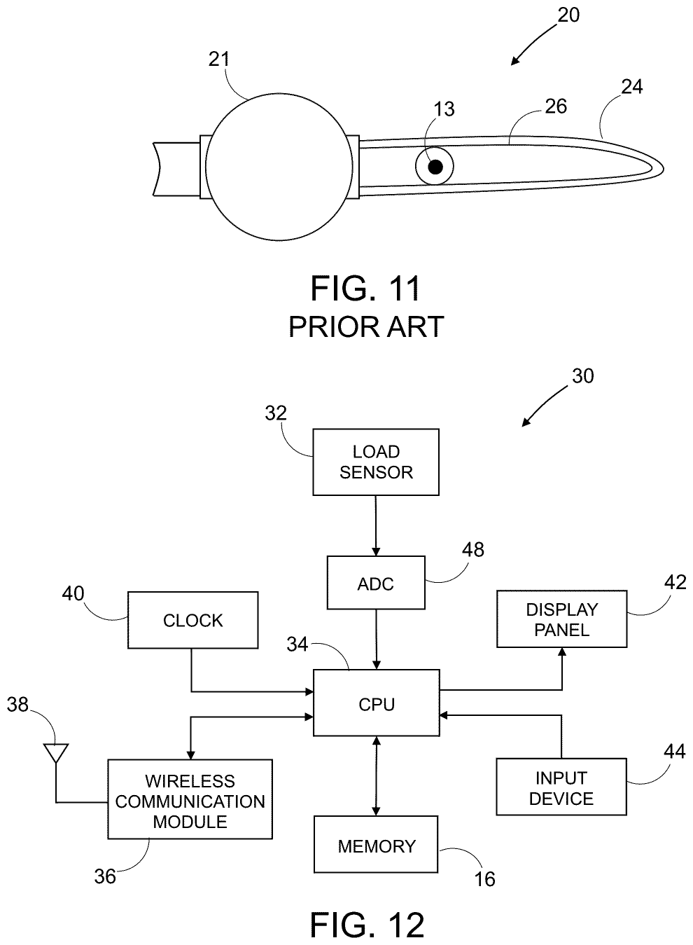

Wristwatch 20 comprises a physiological sensor 13 that produces an analog signal which represents the changing value of a physiological variable (e.g., a cardiovascular variable) of an individual during a time period when the individual is eating. The physiological sensor 13 is connected to a CPU 11 by way of an intervening analog-to-digital converter (not shown in FIG. 1). After analog-to-digital conversion, CPU 11 is programmed to send the physiological data to a wireless communication module 19, which broadcasts radio waves representing the acquired physiological data via an antenna 27. One embodiment of a wristwatch 20 will be described in more detail below with reference to FIG. 11.

Body weight scale 30 comprises a load sensor 32 that produces an analog signal which is proportional to the body weight of an individual who is standing on the scale. The load sensor 32 is connected to a CPU 34 by way of an intervening analog-to-digital converter (not shown in FIG. 1). After analog-to-digital conversion, the digital load sensor data is translated by a CPU 34 into digital data representing body weight as a number of pounds. In addition, the CPU 34 is programmed send the digital body weight data of the weighed individual to the wireless communication module 36, which broadcasts radio waves representing the body weight data via an antenna 27. In accordance with an alternative embodiment, the dieting individual may read his body weight on the display panel of the scale and then manually input his body weight into mobile telephone 50. The CPU 34 is further programmed to maintain a weight log in memory (not shown in FIG. 1), which weight log includes weight information over a period of time for the dieting individual. One embodiment of a body weight scale 30 will be described in more detail below with reference to FIG. 12.

The signals broadcast at different times by the antennas 27 and 38 are detected by an antenna 53 which is connected to wireless communication module 52 of the mobile telephone 50 (provided that the mobile telephone is within range). The mobile telephone 50 further comprises a CPU 54 which is programmed to process the physiological data and body weight data received from wristwatch 20 and body weight scale 30 respectively and then send the processed data to memory 68 for storage. The memory 68 is connected to CPU 54 by way of an intervening MMU 67. The MMU 67 may be configured to receive addresses from CPU 54 and translate those addresses into locations in memory 68. In some embodiments, the MMU 67 may be incorporated inside the CPU 54.

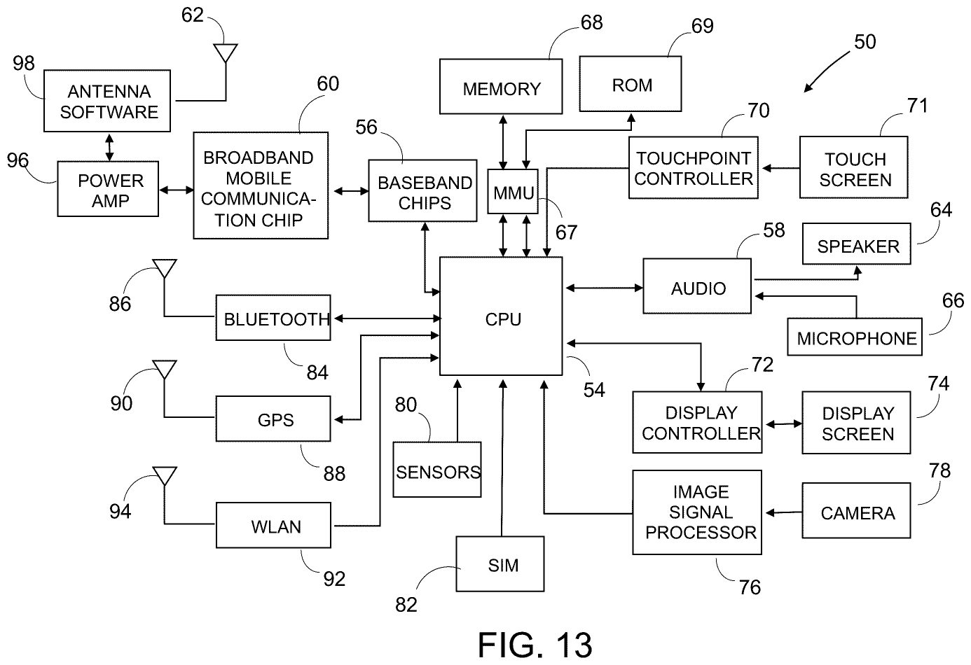

In accordance with the embodiment depicted in FIG. 1, the mobile telephone further comprises a pair of baseband chips 56 and an audio chip 58 which communicate bidirectionally with the CPU 54. The baseband chips 56 are configured to transmit simultaneous voice and data transfer. In well-known manner, baseband chips 56 convert digital signals into analog signals that can be transmitted over cable or phone lines, and convert incoming analog signals back into digital signals so that the CPU 54 can process them. The mobile telephone 50 further includes a broadband mobile communications chip 60 (e.g., 4G or 5G) which is configured to function as a portable modem that enables wireless Internet access delivered through cellular towers to computers and other digital devices. The broadband mobile communications chip 60 generates electric currents which are converted into broadcast radio waves by an antenna 62. One embodiment of a mobile telephone 50 will be described in more detail below with reference to FIG. 13.

In accordance with an alternative embodiment, a mobile device that does not have telephony components (i.e., does not include baseband chips 56 and broadband mobile communications chip 60 seen in FIG. 1) may be used instead of a mobile telephone. Such a mobile device would include a wireless communications module 52, an antenna 53, a CPU 54, and a memory 68. As used herein and in the claims, the term “mobile device” should be construed to include a mobile telephone or other battery-powered mobile device (such as a personal digital assistant).

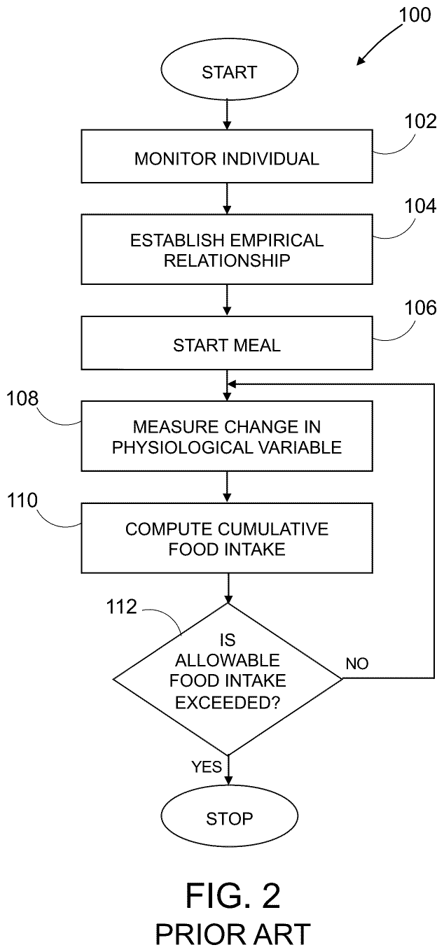

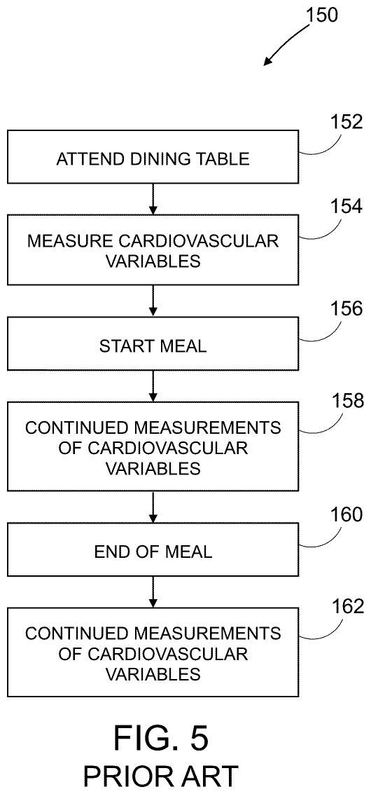

FIG. 2 is a flow diagram showing the principal steps associated with a method 100 for monitoring the food intake of an individual as disclosed in U.S. Pat. No. 5,398,688. In the first stage, the individual is monitored (step 102) in order to establish an empirical relationship for the individual between one or more physiological variables and an amount of food intake (step 104). The manner in which this is done will be described in more detail below with reference to FIG. 5. At this stage, it is sufficient to understand that the empirical relationship permits a change in a physiological variable of the individual to be correlated with a rate and/or amount of food intake. After the dieting individual starts eating (step 106), the actual change in the physiological variable is measured (step 108). By measuring the actual change in the physiological variable during eating after the empirical relationship has been established for the individual, the empirical relationship may then be employed in a reverse manner to estimate (i.e., compute) the cumulative food intake (step 110). (In the alternative, the rate of food intake may be computed.) A determination is then made whether the allowable food intake has been exceeded (step 112). On the one hand, if the allowable food intake has not been exceeded, then the method 100 returns to step 108. On the other hand, if the allowable food intake has been exceeded, then the dieting individual is instructed to stop eating.

It has been reported that the physiological variables most susceptible to defined changes owing to food consumption are first, heart rate and second, stroke volume, i.e. the volume of blood pumped by the heart through the blood vessels per beat as determined by radial artery flow. However, other physiological variables may also be used as a guide to the rate and/or amount of caloric intake. For example, as a person eats, his skin temperature may rise and the change in skin temperature can be measured using a thermistor. Likewise, there are measurable changes in blood pressure and these can be effectively measured by means of a suitable ultrasonic or electronic transducer, e.g., a Doppler ultrasound transducer.

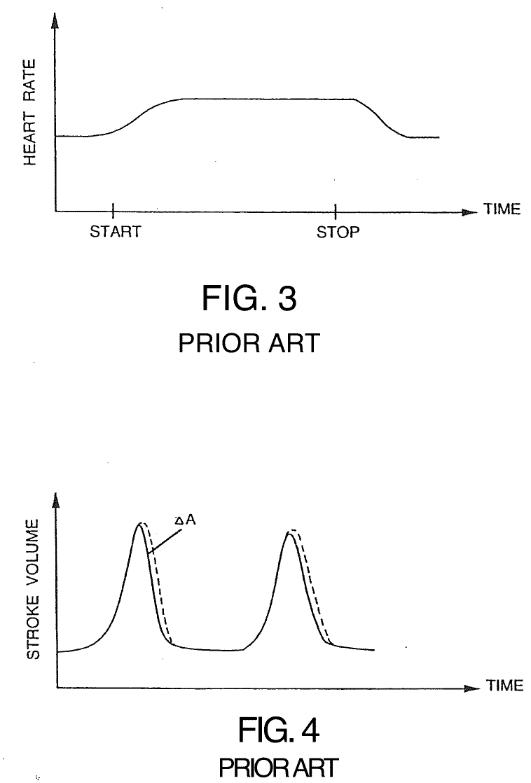

FIG. 3 shows graphically the effect of eating on heart rate. It will be seen that when the individual starts eating, there is a rise in heart rate which settles down to a new, higher level during the course of the meal. After the individual stops eating, the heart rate falls to its original level. In fact, the heart rate may well start to rise even before the actual commencement of the meal, particularly if the individual is anxiously anticipating his meal due to appetite, hunger, or some other reason. Nevertheless, a difference still exists between the heart rate before and after eating, and the magnitude of this difference is related to the amount and rate of ingested food.

FIG. 4 shows graphically the effect of eating on the stroke volume curve. The general shape of FIG. 4 is equally applicable whether the pulse or blood flow rate is being measured, either of which can be accomplished with appropriate instrumentation. In either case, the area under the curve increases consequent to caloric intake and the differential area under the curve ΔA may be used as an empirical relationship for estimating the rate and/or amount of food intake and hence of overall food intake.

In addition to the increase in heart rate and stroke volume, blood pressure also rises shortly after initiation of eating and so may be used as a guide for determining an empirical relationship between change in blood pressure and food intake. Furthermore, regardless of which specific physiological variable or variables is/are employed for the formulation of the empirical relationship, the increments vary among individuals but are particularly related to caloric intake. Assuming a stable emotional status and roughly similar physical surroundings, time of day, and food temperature, intra-individual variations in hemodynamic responses to food depend predominantly on caloric intake and rate of feeding. Thus, in the same individual, a large meal consumed swiftly elicits large increments in heart rate, stroke volume and blood pressure, whereas a small meal consumed casually over a long period of time, results in relatively minor changes. Thus, repetitive quantification of the hemodynamic responses in a given individual under basal (“pre-diet”) conditions generates a reliable range of “dose response” patterns which may be utilized for subsequent programming aimed at caloric restriction and deceleration of feeding rate.

Referring now to FIG. 5, an explanation will be provided how the initial monitoring 150 is performed in order to establish the empirical relationship between the change in the desired physiological variable and food intake. After the individual has sat down at a dining table (step 152) and prior to eating each meal, all of the above-mentioned cardiovascular variables are measured and recorded (step 154). Thereafter, the individual starts eating a pre-measured meal (step 156) and any changes in the corresponding cardiovascular variables are measured and also recorded (step 158). In all respects, the prospective dieter eats at his normal pace and stops eating only when he has finished his meal (step 160). After the meal has ended, any changes in the corresponding cardiovascular variables are again measured and also recorded (step 160).

It should be appreciated that no pressure is put on the prospective dieter, during the initial monitoring phase, to eat less because the whole objective of the initial monitoring phase is to establish an empirical relationship between the measured cardiovascular variables and the specific individual's food intake under normal circumstances. The elapsed time during which the individual was eating is also measured and recorded and the whole cycle is repeated for as many meals as possible during the course of one week or as required, in a controlled environment.

Thus, during the week or weeks of observation, constituting an initial monitoring phase, there will be recorded for each meal all respective changes in the monitored cardiovascular variables prior to the onset of eating and subsequent thereto. There will also be recorded the actual food intake for each meal. It is therefore a simple task to establish an average food intake for each of the meals eaten during the monitoring phase and a corresponding average change in the monitored cardiovascular variables.

This having been done, the standard deviation of the results is determined in order to establish whether the measured averages are sufficiently close to the sampled readings as to form a reliable baseline for future correlation of food intake from the change in the cardiovascular variables of interest.

The initial monitoring phase can be extended over a reasonable period of time in order to establish a reliable baseline. For example, if during the initial monitoring phase, the individual eats only vegetarian meals while, under normal circumstances, he eats meat at least one meal a day, then the results will be skewed and will not provide a good basis for subsequent weight loss. Therefore, the initial monitoring phase is performed over a period of at least one week in order to establish a stable and reliable baseline. During this period, the repeat measurements allow the determination of the “best-fitting cardiovascular variable”, i.e., the variable which is best related to the particular individual's food intake.

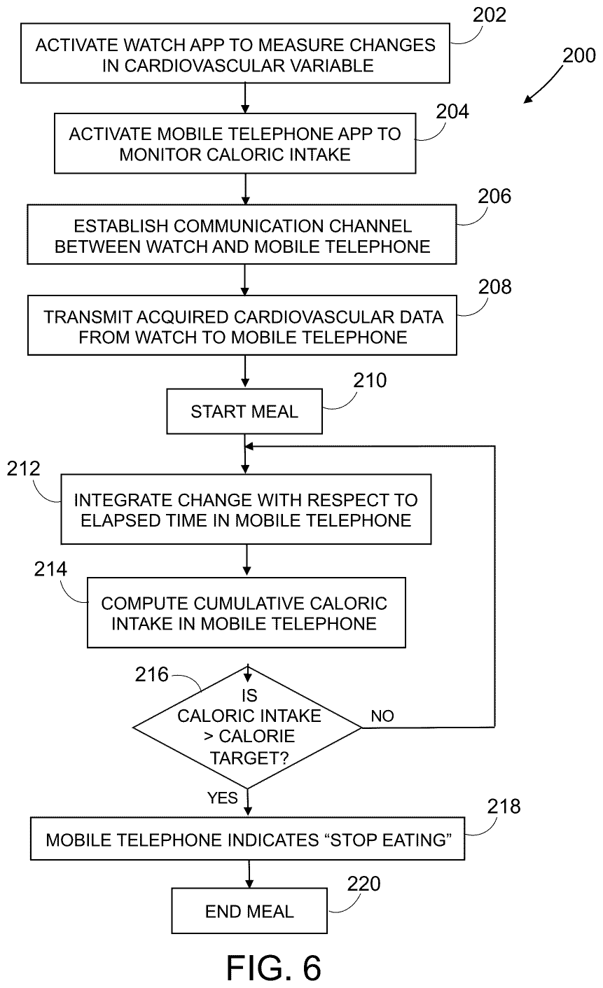

FIG. 6 is a flowchart showing the principal steps associated with a process 200 that monitors the cumulative caloric intake of a dieting individual based on measured changes in a cardiovascular variable in accordance with one embodiment. Before the start of each eating occurrence, the dieting individual first activates a measurement routine for measuring a cardiovascular variable on his wristwatch (step 202). Then the dieting individual activates the caloric intake monitoring routine on his mobile telephone (step 204). As part of this activation process, the dieting individual inputs into the mobile telephone data identifying the type of eating occurrence (breakfast, lunch, dinner, or snack) which is about to begin. In response to the performance of step 204, the mobile telephone establishes a communication channel with the wristwatch (e.g., using Bluetooth) and retrieves the relevant calorie target corresponding to the type of eating occurrence from memory (step 206). Thereafter, the wristwatch 20 transmits the measurement results (hereinafter “acquired cardiovascular data”) to the mobile telephone 50 (step 208) via the respective paired wireless communications modules 19 and 52 (seen in FIG. 1). The dieting individual then starts to eat (step 210). While the dieting individual is eating, the CPU 54 of mobile telephone 50 processes the acquired cardiovascular variable data by integrating the changing cardiovascular variable with respect to elapsed time (step 212) and then computing the cumulative caloric intake (step 214). A determination is then made whether the cumulative caloric intake has exceeded the calorie target for the meal currently in progress (step 216). On the one hand, if the calorie target has not been exceeded, then the method 200 returns to step 212. On the other hand, if the calorie target has been exceeded, then the mobile telephone 50 issues an audio or visual alarm indicating that the dieting individual should stop eating (step 218). The meal ends when the dieting individual stops eating (step 220). This monitoring process may be repeated for each eating occurrence (i.e., breakfast, lunch, dinner, and snacks) until the weight loss target has been achieved.

In addition to the caloric intake monitoring routine, the diet management application installed in the mobile telephone 50 includes a weight loss analysis routine which computes the current weight loss of the dieting individual and then compares the current weight loss to a target weight loss. In the absence of any weight loss, the analysis routine automatically reduces the magnitudes of the calorie targets stored in mobile telephone memory, which reduced calorie targets are then used to monitor subsequent eating occurrences.

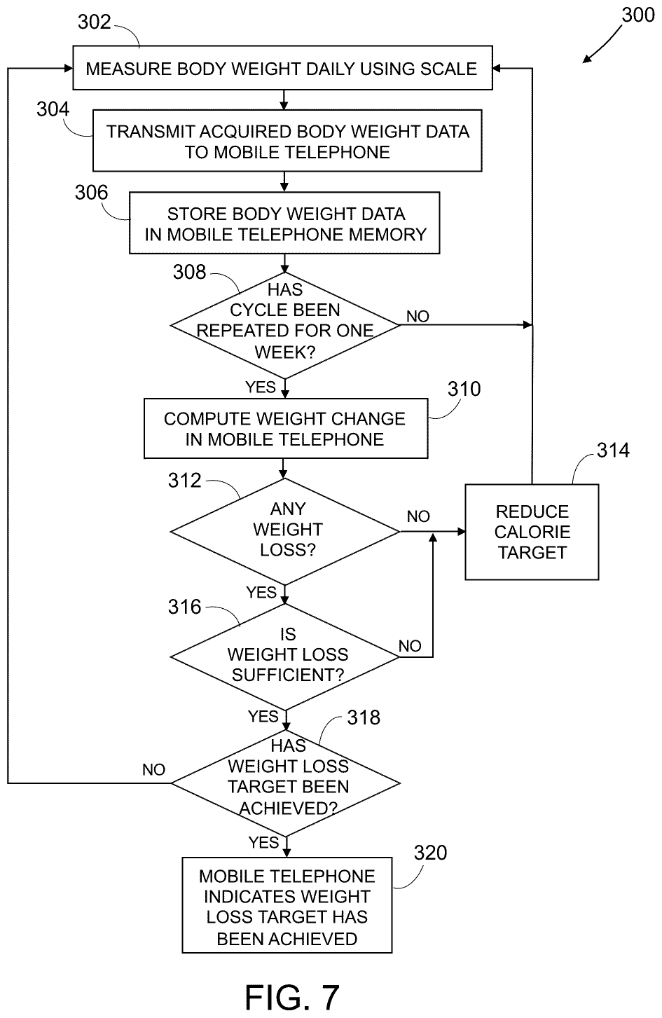

FIG. 7 is a flowchart showing the principal steps associated with an analysis routine 300 that concludes with an adjustment of the calorie target(s) stored in mobile telephone memory based on measured changes in body weight in accordance with one embodiment. The individual's weight is measured and recorded at a specific known time on a daily basis using the body weight scale 30 (step 302). The acquired body weight data is transmitted from the body weight scale 30 to the mobile telephone 50 (step 304) via the respective paired wireless communications modules 36 and 52 (seen in FIG. 1). The mobile telephone 50 stores the received body weight data in its memory (step 306).

In accordance with the embodiment depicted in FIG. 7, the analysis routine 300 is performed automatically by the CPU 54 of mobile telephone 50 on a weekly basis. A determination is made whether the cycle comprising steps 302, 304, and 306 has been repeated for one week (step 308). On the one hand, if the cycle has not been repeated for one week, then the method 300 returns to step 302. On the other hand, if the cycle has been repeated for one week, then the CPU 54 of mobile telephone 50 subtracts the individual's body weight on the last day of the current week from the individual's body weight on the last day of the previous week to determine a difference in the two body weights (step 310). A determination is made whether the weight change is negative, indicating a weight loss (step 312). On the one hand, if there has been no weight loss, then the CPU 54 reduces the magnitudes of the calorie targets stored in mobile telephone memory by a percentage or number of calories which may be function of the magnitude of any weight gain (step 314). The analysis routine then returns to step 302. On the other hand, if there has been weight loss, then the CPU 54 determines whether the weight loss is sufficient to achieve the target weight loss within the scheduled time period or not (step 316). On the one hand, if the weight loss is insufficient to meet the schedule if continued on a weekly basis, then the CPU 54 reduces the calorie targets stored in mobile telephone memory by a percentage or number of calories which may be function of the magnitude of the weight loss (step 314). Again, the analysis routine then returns to step 302. On the other hand, if the weight loss is sufficient to meet the schedule if continued on a weekly basis, then the CPU 54 determines whether the weight loss target has been achieved or not (step 318). On the one hand, if the weight loss target has not been achieved, then the analysis routine returns to step 302. On the other hand, if the weight loss target has been achieved, then the mobile telephone indicates to the dieting individual that the weight loss target has been achieved (step 320).

Optionally, the dieting individual may be notified that the calorie targets have been reduced. In addition, the dieting individual may invoke the sequence comprising steps 310, 312, 314, and 316 by inputting an appropriate command to the mobile telephone. In response to invocation of this sequence, the CPU 54 of mobile telephone 50 may be configured to compute the difference between the individual's current body weight and a body weight target and then notify the individual of that difference either audibly or visually.

If there is no measured weight loss following a week of dieting, then it is clear that the same eating pattern cannot be allowed to prevail indefinitely if a weight loss is to be achieved. In accordance with another embodiment, the time duration of subsequent meals is curtailed relative to the measured and recorded time durations for previous meals of the same category. This will ensure that, providing in all other respects the same eating pattern is maintained, less food will be consumed and this is repeated, as required, until a weekly weight loss is finally recorded. If the measured weight loss is insufficient, then the entire cycle of subsequent meals eaten during the course of a week is still further modified until the measured weight loss is equal to or greater than a preset threshold representing a weekly weight loss target. The CPU 54 of mobile telephone 50 could be configured (i.e., programmed) to monitor the weight loss on a biweekly or monthly basis rather than a weekly basis.

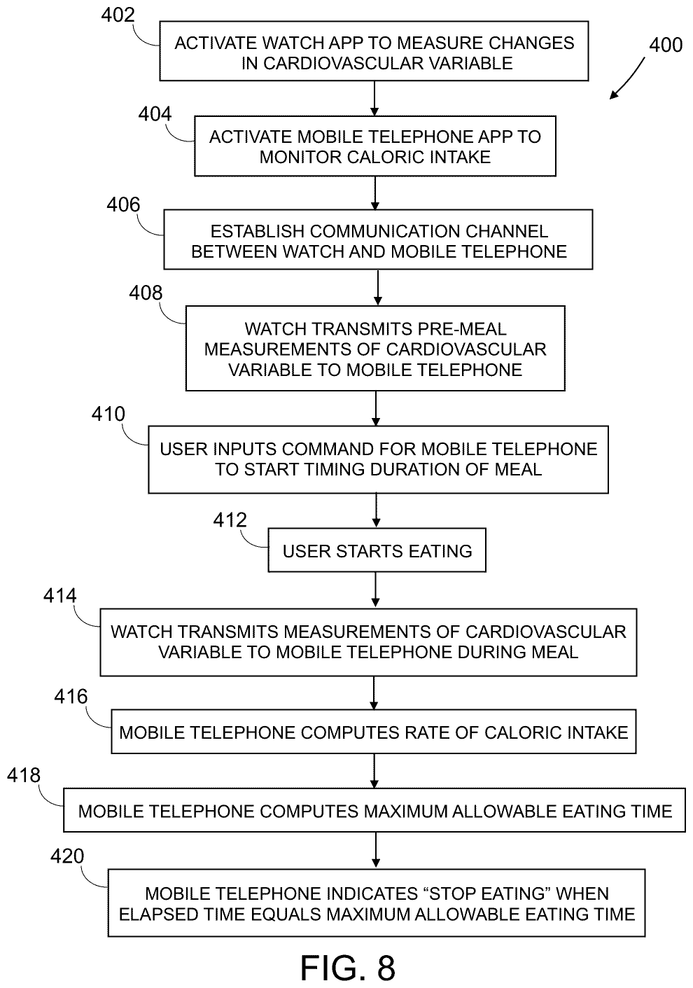

FIG. 8 is a flowchart showing the principal steps associated with a monitoring process 400 that monitors the rate of caloric intake of a dieting individual based on measured changes in a best-fitting cardiovascular variable in accordance with another embodiment. Before the start of each eating occurrence, the dieting individual first activates a measurement routine for measuring a cardiovascular variable on his wristwatch (step 402). Then the dieting individual activates the caloric intake monitoring routine on his mobile telephone (step 404). As part of this activation process, the dieting individual inputs into the mobile telephone data identifying the type of eating occurrence (breakfast, lunch, dinner, or snack) which is about to begin. In response to the performance of step 404, the mobile telephone establishes a communication channel with the wristwatch (e.g., using Bluetooth) and retrieves the relevant calorie target corresponding to the type of eating occurrence from memory (step 406). Thereafter and prior to the start of eating, the wristwatch 20 transmits acquired cardiovascular variable data to the mobile telephone 50 (step 408). Immediately prior to starting to eat, the dieting individual interacts with the mobile telephone 50 by inputting a command to start timing the duration of the eating session (step 410). The dieting individual then starts to eat (step 412). While the dieting individual is eating, the wristwatch 20 continues to transmit acquired cardiovascular variable data to mobile telephone 50 (step 414). The CPU 54 of mobile telephone 50 processes the cardiovascular variable data to determine (i.e., estimate) the rate of caloric intake while also tracking the time elapsed since the start of eating (step 416). More specifically, based on knowledge of the measured change in the cardiovascular variable and also the empirical relationship between the change in cardiovascular variable and caloric intake, the estimated rate of caloric intake may be calculated. The CPU 54 then retrieves the applicable calorie target from memory and computes a maximum allowable eating time by dividing the calorie target by the estimated rate of caloric intake (step 418). When the elapsed time of the eating occurrence becomes equal to or greater than the maximum allowable eating time, the mobile telephone issues an audio or visual alarm indicating that the dieting individual should stop eating (step 420). This monitoring process may be repeated for each eating occurrence until the weight loss target for the dietary program has been achieved.

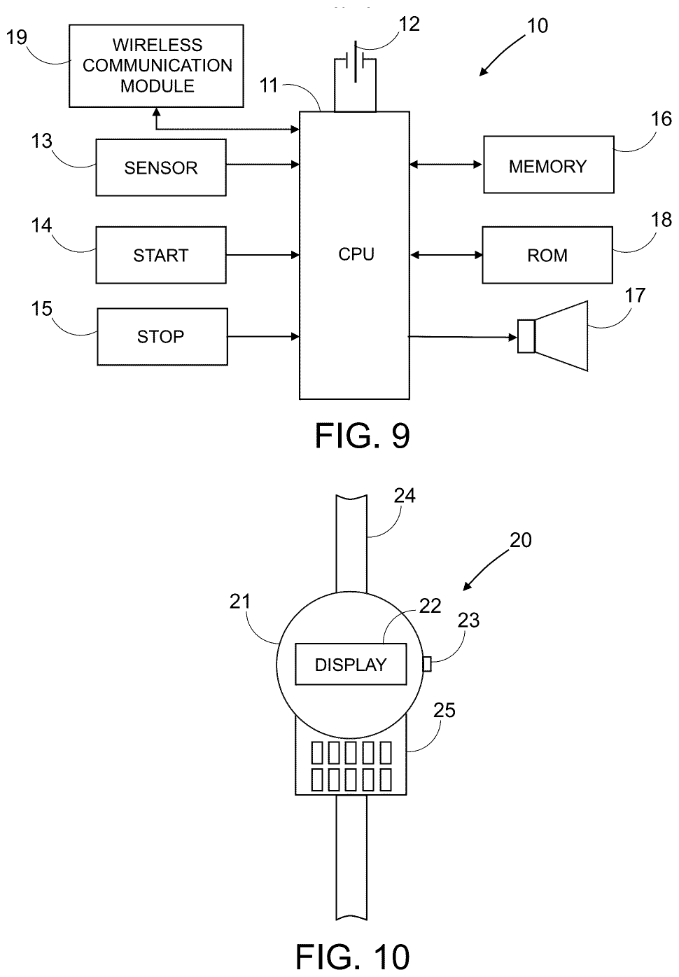

FIG. 9 is a block diagram identifying some components of a wearable instrument 10 which is configured to measure a cardiovascular variable of a dieting individual and then transmit the measurements to a mobile telephone. The instrument 10 may be a wristwatch or some other device which can be strapped to a limb of the dieting individual. The instrument 10 includes a CPU 11 and a physiological sensor 13, which effectively communicate via an analog-to-digital converter (not shown in FIG. 9) that converts the analog output of the physiological sensor 13 into digital samples acceptable to CPU 11. The CPU 11 operates at a predetermined clock frequency derived by means of a quartz crystal 12. The instrument 10 may be strapped to the dieting individual's wrist in a manner such that the physiological sensor 13 is located on the individual's radial artery for sensing the individual's heart rate or stroke volume. In accordance with one proposed implementation, the physiological sensor 13 is a Doppler ultrasound transducer. START and STOP switches 14 and 15, respectively, are coupled to the CPU 11 for activating and deactivating, respectively, the physiological sensor 13 for measuring a physiological variable, such as a cardiovascular variable. Also connected to the CPU 11 is a memory 16 for storing therein a time history of cardiovascular variables measured by the physiological sensor 13 whereby, after extended use of the instrument 10, the average, steady-state values of the cardiovascular variables prior to the onset of eating may be determined by reading the time history from memory 16 even without measuring the cardiovascular variable discretely prior to the onset of eating. This also overcomes any tendency to increased cardiovascular activity even prior to eating owing to anxiousness, hunger, appetite, and so forth. The instrument 11 further includes read only memory 18, in which the operating system is stored. A small loudspeaker 17 is coupled to the CPU 11 for enabling audible annunciations of interest to the dieting individual, such as error messages or instructions. The instrument may further include a battery (not shown in FIG. 9) that is used to store and provide power to the other components of the instrument. The battery may be a rechargeable power supply that is configured to provide power to the instrument.

Preferably, the instrument 10 is in the form of a wristwatch and includes conventional watch functions so that, when the instrument 10 is not being used for the purpose of measuring cardiovascular variables, it can be used as a normal wristwatch. In use, the individual straps the instrument 10 to his wrist so that the physiological sensor 13 overlies the radial artery. When the START switch 14 is pressed, the physiological sensor 13 is activated to measure the cardiovascular variable at the site where the sensor is located. The physiological sensor 13 outputs an analog signal which is converted to digital samples representing cardiovascular variable data. The digital samples and the clock signal are continuously received by the CPU 11. The CPU 11 executes operations in accordance with programming code stored in memory 16, including correlating received digital samples with the respective times at which the measurements were acquired. The CPU 11 continuously outputs time-stamped digital samples representing cardiovascular variable data to the wireless communication module 19, which then broadcasts the time-stamped digital samples via an antenna (not shown in FIG. 9, but see antenna 27 in FIG. 1).

FIG. 10 shows pictorially a wristwatch 20 having a dial 21, a display window 22, a MODE switch 23, and a strap 24 for attaching the wristwatch 20 to the individual's wrist and itself comprising a sensor (not shown in FIG. 10) for overlying the radial artery so as to detect the required cardiovascular variable or variables. The wristwatch 20 shown in FIG. 10 also includes a keyboard 25 for allowing the dieting individual to interface with a CPU installed within the housing of wristwatch 20 and not visible in FIG. 10. For example, the keyboard 25 could be used to enable the dieting individual to pair the wireless communication module inside wristwatch 20 with the wireless communication module inside his mobile telephone to establish a communication channel that employs Bluetooth or other communication protocol (e.g., by typing in the word PAIR). In accordance with the embodiment depicted in FIG. 10, the measurement routine is activated by pressing the MODE switch 23. At the end of the meal, pressing the MODE switch 23 a second time after the meal has ended deactivates the measurement routine.

FIG. 11 shows pictorially a detail of a reverse view of the wristwatch 20 from which it will be seen that the strap 24 is provided on its lower surface with a peripheral track 26. The physiological sensor 13 is slidably fixed to the strap 24 so as to allow movement of the physiological sensor 13 within the peripheral track 26. During use, prior to attaching the wristwatch 20 to the individual's wrist, the dial 21 is positioned and the physiological sensor 13 is moved around the individual's wrist relative to the dial 21 so as to allow positioning of the physiological sensor 13 in proximity to the individual's radial artery.

FIG. 12 is a block diagram identifying some components of a body weight scale 30 in accordance with one embodiment. The body weight scale 30 comprises a load sensor 32, a CPU 34, a wireless communications module 36, an analog-to-digital converter (ADC) 48, a memory 16, a display panel 42, an input device 44, and other components not shown, such as a display controller which controls the display panel to display body weight data output by CPU 34 in response to the dieting individual stepping on the scale. The input device 44 may include a keyboard, mouse, touch screen, or other device to allow a user to input information into the body weight scale 50. Additionally, body weight scale 50 includes a clock 40 to determine weigh-in date and time for a particular scale use. A battery for powering the electrical components of body weight scale 30 is not shown in FIG. 11.

The load sensor 32 produces an analog signal representing the individual's body weight, which analog signal is converted to digital load sensor data by the ADC 48 that is sent to the CPU 34. The digital load sensor data is translated by CPU 34 into digital data representing body weight as a number of pounds. The CPU 34 sends the digital body weight data to the display controller for display on the display panel 42. In addition, the CPU 34 is programmed to send the digital body weight data of the weighed individual to the wireless communication module 36, which broadcasts radio waves representing the body weight data via an antenna 27. The wireless communication module 36 provides data communication connectivity to paired devices within broadcast range, such as the mobile telephone 50 depicted in FIG. 1.

The CPU 34 is further programmed to maintain a weight log in memory 16, which weight log includes weight information over a period of time for the dieting individual. More specifically, memory 16 stores data including a user identifier (ID) to uniquely identify a scale user and a weight log uniquely belonging to the user identified by the user ID. The weight log includes information such as the individual's weight and weigh-in date and time for each weighing event. The weight log may be updated with a new log entry every time a particular individual uses the body weight scale 50, for example, daily or weekly.

FIG. 13 is a block diagram identifying some components of a mobile telephone 50 in accordance with one embodiment. The mobile telephone 50 may be a commercially available off-the-shelf product incorporating standard hardware or software. The hardware includes a CPU 54, memory 68, and ROM 69. The memory 68 (e.g., random access memory) and the ROM 69 are both connected to CPU 54 by an intervening MMU 67, as previously described with reference to FIG. 1. To enable the method for managing a dietary program disclosed herein, diet management application software is downloaded and stored in memory 68. The acquired physiological data and body weight data are stored in memory 68, which may include memory chips of different types. The operating system of the mobile telephone 50 is stored in ROM 69.

The diet management application stored in memory 68 is executable by the CPU 54. The diet management application includes a user-selectable caloric intake monitoring routine that is capable of monitoring the physiological variable data received from the wristwatch 20 (see FIG. 1) during an eating occurrence and a user-selectable weight loss analysis routine that is capable of computing the amount of weight loss based on the body weight data received from the body weight scale 30 (see FIG. 1) subsequent to a weighing event. The dieting individual can invoke execution of the diet management application by inputting a command to the CPU 54 via a touch screen 71, which is connected to CPU 54 by means of an intervening touchpoint controller 70.

The caloric intake data and body weight data can be displayed on a display screen 74, which is connected to CPU 54 by means of an intervening display controller 72. The user interface of mobile telephone 50 further includes a speaker 64 and a microphone 66 which are connected to CPU 54 via an intervening audio chip 58. The audio chip 58 is configured to convert analog signals received from microphone 66 into digital signals sent to CPU 54 and to convert digital signals received from CPU 54 into analog signals sent to speaker 64. The mobile telephone 50 also includes a camera 78, which is connected to CPU 54 by means of an intervening image signal processor 76. The image signal processor 76 processes raw image data output by camera 78 into a high-quality digital image.

The mobile telephone 50 depicted in FIG. 13 further includes means for enabling broadband mobile communications, which include a pair of baseband chips 56, broadband communications chip 60, power amplifier 96, antenna software 98, and antenna 62. The baseband chips 56 are configured to transmit simultaneous voice and data transfer. In well-known manner, baseband chips 56 convert digital signals into analog signals that can be transmitted over cable or phone lines, and convert incoming analog signals back into digital signals so that the CPU 54 can process them. The broadband mobile communications chip 60 (e.g., 4G or 5G) is configured to function as a mobile broadband modem. A mobile broadband modem, also known as wireless modem or cellular modem, is a type of modem that allows a personal computer or a router to receive wireless Internet access via a mobile broadband connection instead of using telephone or cable television lines. In transmission, the broadband mobile communications chip 60 supplies an electric current to the antenna's terminals, and the antenna 62 radiates (i.e., broadcasts) the energy from the current as electromagnetic waves (radio waves). In reception, the antenna 62 intercepts some of the power of a radio wave in order to produce an electric current at its terminals that is amplified by the power amplifier 96. (As used herein, the term “modem” means a modulator-demodulator that converts data from a digital format into a format suitable for an analog transmission medium such as telephone or radio. A modem transmits data by modulating one or more carrier wave signals to encode digital information, while the receiver demodulates the signal to recreate the original digital information.)

The mobile telephone 50 depicted in FIG. 13 further includes means for enabling short-range wireless communications in the form of a Bluetooth chip 86 that is connected to the CPU 54 and to an antenna 86. The antenna 86 detects radio waves broadcast by the wristwatch 20 and the body weight scale 30 when the mobile telephone 50 is within range of either instrument. The Bluetooth chip 86 contains the processing units and circuits needed for Bluetooth communication. In the most widely used mode, transmission power is limited to 2.5 milliwatts, giving Bluetooth a short range of up to 10 meters (33 ft). It employs ultra-high-frequency radio waves in the 2.4-GHz range. The CPU 54 is programmed to run the diet management application which is stored in memory 68. CPU 54 process the physiological data and body weight data received from Bluetooth chip 86 and then sends the processed data to memory 68 for storage.

In accordance with one embodiment, the CPU 54 of mobile telephone 50 executes a caloric intake monitoring routine that converts the physiological variable data received from the wristwatch 20 into caloric intake data during each eating occasion. The caloric intake data in turn is stored in memory 68. The memory 68 also stores body weight data received from the body weight scale 30. In addition, the CPU 54 of mobile telephone 50 may be selectively activated to execute an analysis routine that computes the amount of weight loss during a most recent interval of time. In response to a determination that the computed weight loss is less than a preset weight loss target, the CPU 54 is capable of reducing the magnitudes of the calorie targets stored in memory 68 for each type of eating occurrence, which revised calorie targets will be used to monitor succeeding eating occurrences.

The mobile telephone is also equipped with a second short-range wireless communication circuitry, namely, a WLAN chip 92, which is connected to an antenna 94. The WLAN chip 92 incorporates the processing units and circuits needed to enable the mobile telephone 50 to communicate with a local area network having a wireless access point and a wireless router. The WLAN chip 92 enables the mobile telephone 50 to connect to the Internet via the local area network. For example, the mobile telephone 50 may transmit the caloric intake data and the body weight data via the Internet to a remote device used by a dietician who is responsible for overseeing the individual's dietary program.

Although not pertinent to the capability of the mobile telephone 50 to manage the dietary program of an individual, the mobile telephone 50 may also include: a Global Positioning System chip 88 which is connected to CPU 54 and to an antenna 90; sensors 80 (such as a photodetector, a temperature sensor, a light or optical sensor, an atmospheric pressure sensor, a humidity sensor, a magnet, a gyroscope, an accelerometer, and so on) which are connected to CPU 54 by respective ADCs (not shown in FIG. 13); and a Subscriber Identity Module (SIM) entity 82 which is connected to the CPU 54 for the purpose of executing SIM application and/or otherwise implement SIM functionality. The mobile telephone 50 may further include a battery (not shown in FIG. 13) that is used to store and provide power to the other components of the telephone.

While systems and methods for monitoring caloric intake have been described with reference to various embodiments, it will be understood by those skilled in the art that various changes may be made and equivalents may be substituted for elements thereof without departing from the scope of the teachings herein. In addition, many modifications may be made to adapt the teachings herein to a particular situation without departing from the scope thereof. Therefore it is intended that the claims not be limited to the particular embodiments disclosed herein.

The embodiments disclosed above use one or more computer systems. As used in the claims, the term “computer system” should be broadly construed to include each of the following alternative implementations: a single central processing unit, a microcontroller, multiple processing or computing chips that communicate with each other, a reduced instruction set computer processor, an application-specific integrated circuit, a programmable logic circuit, a field-programmable gated array, a digital signal processor, and/or any other circuit or processing device capable of executing the functions described herein.

Certain systems, apparatus, applications or processes have been described herein as including a number of modules. A module may be a unit of distinct functionality that may be implemented in software, hardware, or combinations thereof, except for those modules which are preferably implemented as hardware or firmware. When the functionality of a module is performed in any part through software, the module can include a non-transitory tangible computer-readable storage medium.

The flowcharts and block diagrams illustrate the architecture, functionality, and operation of one possible implementation of apparatus and methods in accordance with one exemplary embodiment. In this regard, each computational block in the flowcharts or block diagrams may represent a module, segment, function, and/or a portion of an operation or step. For example, one or more of the blocks may be implemented as program code, in hardware, or a combination of the program code and hardware. When implemented in hardware, the hardware may, for example, take the form of integrated circuits that are manufactured or configured to perform one or more operations in the flowcharts or block diagrams.

The methods described and claimed herein may include steps encoded as executable instructions embodied in a non-transitory tangible computer-readable storage medium, including, without limitation, a storage device and/or a memory device. Such instructions, when executed by a processing or computer system (e.g., a CPU), cause the system device to perform at least a portion of the methods described herein.

In the method claims appended hereto, any alphabetic ordering of steps is for the sole purpose of enabling subsequent short-hand references to antecedent steps and not for the purpose of limiting the scope of the claim to require that the method steps be performed in alphabetic order.

As used in the claims, the term “means for indicating” should be construed to include a speaker that generates an audible signal, a display panel that generates a visual signal, and structural equivalents thereof (such as a light source).

1. A wireless network comprising a first instrument and a mobile device, wherein:
the mobile device comprises a first central processing unit, a memory connected to the first central processing unit, a first wireless communication module connected to the first central processing unit, and a first antenna connected to the first wireless communication module;
the first instrument comprises a second central processing unit, a first analog-to-digital converter connected to the second central processing unit, a physiological sensor connected to the first analog-to-digital converter, a second wireless communication module connected to the second central processing unit and paired with the first wireless communication module, and a second antenna connected to the second wireless communication module;
the second central processing unit is programmed to cause digital physiological variable data derived from analog data output by the physiological sensor to be broadcast by the second wireless communication module during an eating occurrence of a dieting individual; and
the first central processing unit is programmed to convert the digital physiological variable data into digital caloric intake data in accordance with an empirical relationship of a physiological variable to caloric intake stored in the memory.

2. The wireless network as recited in claim 1, wherein the physiological variable is a cardiovascular variable.

3. The wireless network as recited in claim 2, wherein the cardiovascular variable is selected from a group comprising heart rate, stroke volume, and blood pressure.

4. The wireless network as recited in claim 1, wherein the first instrument is a wristwatch.

5. The wireless network as recited in claim 1, wherein:
the mobile device further comprises means for indicating; and
the first central processing unit is further programmed to accumulate the caloric intake and then cause the means for indicating to indicate that the dieting individual should stop eating when a cumulative caloric intake becomes equal to or greater than a calorie target stored in the memory.

6. The wireless network as recited in claim 1, wherein the mobile device further comprises means for indicating and the first central processing unit is further programmed to:
measure an elapsed time since a start of eating;
compute a rate of caloric intake;
compute a maximum allowable eating time based on a calorie target stored in the memory and the rate of caloric intake; and
cause the means for indicating to indicate that the dieting individual should stop eating when the elapsed time becomes equal to or greater than maximum allowable eating time.

7. The wireless network as recited in claim 5, further comprising a second instrument, wherein:
the second instrument comprises a third central processing unit, a second analog-to-digital converter connected to the third central processing unit, a load sensor connected to the second analog-to-digital converter, a third wireless communication module connected to the third central processing unit and paired with the first wireless communication module, and a third antenna connected to the third wireless communication module;
the third central processing unit is programmed to cause digital body weight data derived from analog data output by the load sensor to be broadcast by the third wireless communication module; and
the first central processing unit is further programmed to:
store the digital body weight data in the memory of the mobile device;
compute an amount of weight loss during a most recent interval of time;
determine that the computed weight loss is less than a preset weight loss target stored in the memory of the mobile device;
reduce a magnitude of the calorie target to produce a revised calorie target; and
use the revised calorie target to monitor caloric intake during a subsequent eating occurrence.

8. The wireless network as recited in claim 7, wherein the first instrument is a wristwatch and the second instrument is a body weight scale.

9. The wireless network as recited in claim 7, wherein the first central processing unit is further programmed to compute a total weight loss using the digital body weight data and to cause the means for indicating to indicate that the dieting individual has successfully completed a dietary program when the total weight loss becomes equal to a weight loss target stored in the memory.

10. A mobile device comprising:
a central processing unit;
a memory connected to the central processing unit;
a wireless communication module connected to the central processing unit; and
an antenna connected to the wireless communication module,
wherein the central processing unit is programmed to convert digital physiological variable data received via the wireless communication module into digital caloric intake data during an eating occurrence of a dieting individual in accordance with an empirical relationship of a physiological variable to a caloric intake stored in the memory.

11. The mobile device as recited in claim 10, wherein the physiological variable is a cardiovascular variable.

12. The mobile device as recited in claim 11, wherein the cardiovascular variable is selected from a group comprising heart rate, stroke volume, and blood pressure.

13. The mobile device as recited in claim 10, further comprising means for indicating, wherein the central processing unit is further programmed to accumulate the caloric intake during the eating occurrence and then cause the means for indicating to indicate that the dieting individual should stop eating when a cumulative caloric intake becomes equal to or greater than a calorie target stored in the memory.

14. The mobile device as recited in claim 10, further comprising means for indicating, wherein the central processing unit is further programmed to:
measure an elapsed time since a start of eating;
compute a rate of caloric intake;
compute a maximum allowable eating time based on a calorie target stored in the memory and the rate of caloric intake; and
cause the means for indicating to indicate that the dieting individual should stop eating when the elapsed time becomes equal to or greater than the maximum allowable eating time.

15. The mobile device as recited in claim 13, wherein the central processing unit is further programmed to:
store the digital body weight data in the memory of the mobile device;
compute an amount of weight loss during a most recent interval of time;
determine that the computed weight loss is less than a preset weight loss target stored in the memory of the mobile device;
reduce a magnitude of the calorie target to produce a revised calorie target; and
use the revised calorie target to monitor caloric intake during a subsequent eating occurrence.

16. A method for monitoring caloric intake during an eating occurrence using a mobile device, the method comprising:
storing an empirical relationship of a physiological variable to a caloric intake of a dieting individual in a memory of the mobile device;
activating a measurement routine to measure a cardiovascular variable of the dieting individual during eating using a first instrument;
activating a caloric intake monitoring routine on the mobile device;
establishing a first communication channel between the first instrument and the mobile device;
transmitting cardiovascular variable data from the first instrument to the mobile device via the first communication channel while the dieting individual is eating;
processing the cardiovascular variable data in the mobile device by integrating the cardiovascular variable with respect to elapsed time during the eating occurrence;
converting the cardiovascular variable data into caloric intake data in accordance with the empirical relationship;
determining that a caloric intake has exceeded a calorie target; and
issuing an audio or visual alarm from the mobile device indicating that the dieting individual should stop eating.

17. The method as recited in claim 16, further comprising:
accumulating the caloric intake during the eating occurrence; and
issuing the audio or visual alarm when a cumulative caloric intake becomes equal to or greater than the calorie target.

18. The method as recited in claim 16, further comprising:
measuring an elapsed time since a start of eating;
computing a rate of caloric intake;
computing a maximum allowable eating time based on the calorie target and the rate of caloric intake; and
issuing the audio or visual alarm when the elapsed time becomes equal to or greater than the maximum allowable eating time.

19. The method as recited in claim 16, further comprising:
activating a measurement routine to measure a body weight of the dieting individual using a second instrument;
establishing a second communication channel between the second instrument and the mobile device;
transmitting body weight data from the second instrument to the mobile device via the second communication channel;
storing the digital body weight data in a memory of the mobile device;
computing an amount of weight loss during a most recent interval of time;
determining that the computed weight loss is less than a preset weight loss target;
reducing a magnitude of the calorie target to produce a revised calorie target; and
using the revised calorie target to monitor caloric intake during a subsequent eating occurrence.

20. The method as recited in claim 19, wherein the first instrument is a wristwatch and the second instrument is a body weight scale.
